(19) United States
(12) Patent Application Publication
(10) Pub. No.: US 2006/0183707 A1
(43) Pub. Date: Aug. 17, 2006

(54) Production method of 2'-deoxyguanosine compound

(75) Inventors: Toshihiro Nishikawa, Kawasaki-shi (JP); Tomoyuki Onishi, Kawasaki-shi (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: AJINOMOTO CO. INC., Tokyo (JP)

(21) Appl. No.: 11/335,640

(22) Filed: Jan. 20, 2006

(30) Foreign Application Priority Data
Jan. 21, 2005 (JP) 014804/2005

(51) Int. Cl. A61K 31/7076; C07H 19/16

(52) U.S. Cl. 514/45; 536/27.21

(57) ABSTRACT

2′-Deoxyguanosine compounds represented by formula (2), may be prepared by desulfurizing an 8,2′-anhydro-8-mercapto-9-β-arabinofuranosylguanine compound represented by formula (1) in a solvent in the presence of a base using nickel or a nickel alloy. 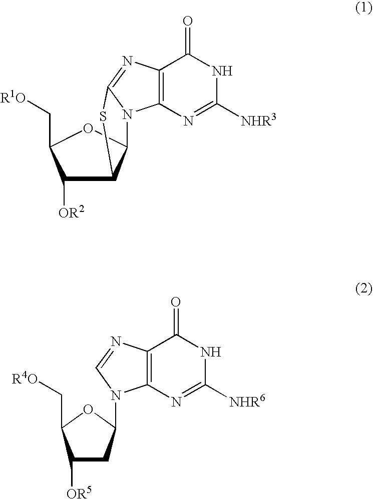
wherein R¹, R², R⁴, and R⁵ are each independently a hydrogen atom or a hydroxyl-protecting group, and R³ and R⁶ are hydrogen atoms or amino-protecting groups.

### CROSS REFERENCES TO RELATED APPLICATIONS

This application claims priority to Japanese Patent Application No. 014804/2005 filed on Jan. 21, 2005, the content of which is hereby incorporated by reference in its entirety.

### BACKGROUND OF THE INVENTION

1. Field of the Invention

The present invention relates to methods of producing 2′-deoxyguanosine compounds. More particularly, the present invention relates to methods of producing 2′-deoxyguanosine compounds which are useful as starting materials for the production of antisense drugs.

2. Discussion of the Background

All biological organisms have genes, biopolymers, and the genes transmit genetic information to the living organisms by being transcribed into mRNA and then translated into a protein. In the case of a disease caused by a genetic factor, it is considered that the disease can be treated by controlling expression of the protein by blocking the flow of genetic information of the pathogenic gene. In recent years, an antisense therapy that base sequence-specifically suppresses expression of such pathogenic genes has been drawing attention. The antisense drug used for such therapy consists of an oligonucleotide and, as a starting material for the production of such an oligonucleotide, deoxynucleoside is known (see, Follmann H., Chem. Soc. Rev., 2004, vol. 33, pp. 225-233).

As a production method of such a deoxynucleoside, for example, the following method which uses an 8,2′-anhydro-8-mercapto-9-β-arabinofuranosylpurine derivative as a starting material has been reported. Ikehara M. et al., Chem. Pharm. Bull., 1966, vol. 15(1), pp. 94-100, disclose production methods of 2′-deoxyadenosine, in which 8,2′-anhydro-8-mercapto-9-β-arabinofuranosyladenine is desulfurized in the presence of about 700 mass % of Raney-nickel in an aqueous solution to give 2′-deoxyadenosine in a yield of 33-40%. In addition, Yamazaki A. et al., Chem. Pharm. Bull., 1973, vol. 21(5), pp. 1143-1146 discloses a production method of 2′-deoxyinosine, in which 8,2′-anhydro-8-mercapto-9-β-arabinofuranosylhypoxanthine is desulfurized in the presence of 6.7 ml of Raney-nickel per 1 g of 8,2′-anhydro-8-mercapto-9-β-arabinofuranosylhypoxanthine, a starting material, in an aqueous solution to give 2′-deoxyinosine in a yield of 60%. Moreover, Ogilvie K. K. et al., Can. J. Chem., 1972, vol. 50, pp. 1100-1104, and Kaneko M. et al., Chem. Pharm. Bull., 1972, vol. 20(3), pp. 635-637 disclose production methods of 2′-deoxyguanosine, in which 8,2′-anhydro-8-mercapto-9-β-arabinofuranosylguanine was desulfurized in the presence of Raney-nickel in an aqueous solution merely to confirm the presence of 2′-deoxyguanosine by thin layer chromatography (TLC), without description of the amount of Raney-nickel used and yield.

Thus, there remains a need for an improved method of producing 2′-deoxyguanosine compounds, which is suitable for industrial implementation.

### SUMMARY OF THE INVENTION

The present inventors have considered the production of 2′-deoxyguanosine by reference to the descriptions of the above-mentioned references, only to discover the problem that the desulfurization reaction of 8,2′-anhydro-8-mercapto-9-β-arabinofuranosylguanine with Raney-nickel hardly proceeds.

Accordingly, it is one object of the present invention to provide novel methods for producing 2′-deoxyguanosine compounds.

It is another object of the present invention to provide novel methods of producing 2′-deoxyguanosine compounds, which are economical and efficient.

It is another object of the present invention to provide novel methods of producing 2′-deoxyguanosine compounds, which are suitable for industrial production.

These and other objects, which will become apparent during the following detailed description, have been achieved by the inventors' discovery that one of the causes of the insufficient yield by conventional production methods of 2′-deoxyguanosine is the solubility of 8,2′-anhydro-8-mercapto-9-β-arabinofuranosylguanine in the reaction system. They have further studied based on that finding and found that a 2′-deoxyguanosine compound can be synthesized in a high yield by desulfurization reaction of an 8,2′-anhydro-8-mercapto-9-β-arabinofuranosylguanine compound in the presence of nickel or a nickel alloy, a solvent, and a base. Particularly, by the use of a nickel-aluminum alloy for the reaction, the amount of a nickel reagent to be used can be markedly reduced as compared to the use of Raney-nickel, based on which finding the present invention has been completed.

Accordingly, the present invention provides the following:

(1) A method of producing a 2′-deoxyguanosine compound represented by the following formula (2) 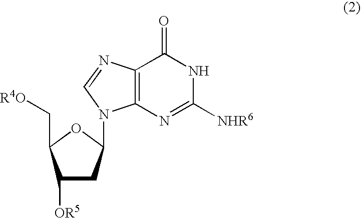
wherein R⁴ and R⁵ are each independently a hydrogen atom or a hydroxyl-protecting group and R⁶ is a hydrogen atom or an amino-protecting group, which comprises desulfurizing an 8,2′-anhydro-8-mercapto-9-β-arabinofuranosylguanine compound represented by the following formula (1) 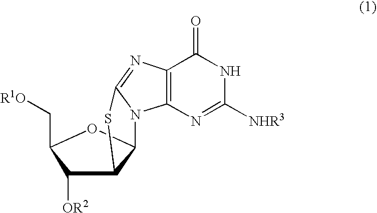
wherein R¹ and R² are each independently a hydrogen atom or a hydroxyl-protecting group and R³ is a hydrogen atom or an amino-protecting group, in the presence of nickel or a nickel alloy, a solvent, and a base.

(2) A method of producing a 2′-deoxyguanosine compound represented by the following formula (3) 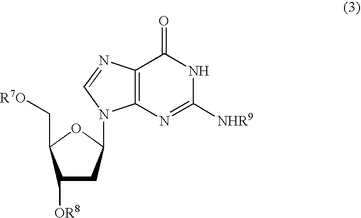
wherein R⁷ and R⁸ are each independently a hydrogen atom or a hydroxyl-protecting group, R⁹ is a hydrogen atom or an amino-protecting group, provided that R⁷, R⁸, and R⁹ are not simultaneously hydrogen atoms, which comprises obtaining a 2′-deoxyguanosine compound represented by the formula (2) according to the production method of the above-mentioned (1), and when one or more of R⁴, R⁵, and R⁶ of the compound are hydrogen atoms, replacing one or more of the hydrogen atoms with protecting group(s).

(3) The production method of the above-mentioned (1), wherein R¹, R², R³, R⁴, R⁵ and R⁶ are hydrogen atoms.

(4) The method of the above-mentioned (3), which further comprises a step of replacing hydrogen atoms for R⁴, R⁵, and R⁶ with protecting groups.

(5) The method of any of the above-mentioned (1) to (4), wherein the solvent is water.

(6) The method of any of the above-mentioned (1) to (5), wherein the desulfurization is carried out in a liquid which comprises a base and a solvent and which has a pH of not less than 11.

(7) The method of any of the above-mentioned (1) to (6), wherein the nickel or nickel alloy is added in an amount of 50 to 1500 mass % of the total mass of the 8,2′-anhydro-8-mercapto-9-β-arabinofuranosylguanine compound.

(8) The method of any of the above-mentioned (1) to (7), wherein the desulfurization reaction is carried out at a temperature of 40 to 100° C.

(9) The method of any of the above-mentioned (1) to (8), wherein the nickel alloy is a nickel-aluminum alloy.

### DETAILED DESCRIPTION OF THE PREFERRED EMBODIMENTS

The method of producing the 2′-deoxyguanosine compound of the present invention is described in the following.

The method of producing the 2′-deoxyguanosine compound of the present invention comprises, as shown in the following reaction scheme, desulfurizing an 8,2′-anhydro-8-mercapto-9-β-arabinofuranosylguanine compound represented by the following formula (1) in a solvent, in the presence of a base, using a nickel or nickel alloy to give a 2′-deoxyguanosine compound represented by the following formula (2), as shown in Scheme I:

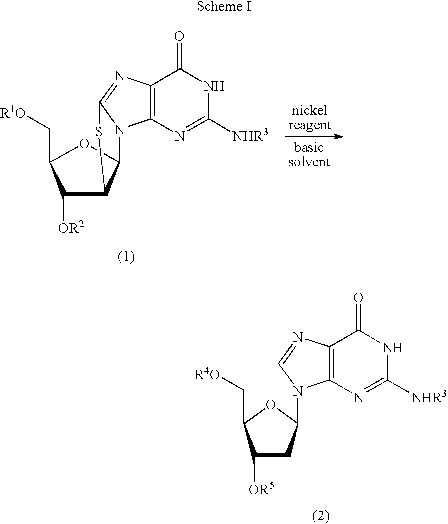

In the above-mentioned production method, when a nickel-aluminum alloy (Ni—Al alloy) is used as the nickel reagent, the Ni—Al alloy is developed to Raney-nickel in a solvent in the presence of a base during a desulfurization reaction. Then, an 8,2′-anhydro-8-mercapto-9-β-arabinofuranosylguanine compound is desulfurized by the reducibility of the Raney-nickel to give a 2′-deoxyguanosine compound. In this case, the amount of the nickel reagent to be used can be markedly reduced and the production cost can be decreased. Moreover, since preparation of Raney-nickel before the desulfurization reaction becomes unnecessary, a 2′-deoxyguanosine compound can be safely produced. The present inventors know that, when the desulfurization reaction is carried out using Raney-nickel prepared before desulfurization reaction in an aqueous solution in the presence of a base, the Raney-nickel needs to be added in an amount of not less 1500 mass % per 8,2′-anhydro-8-mercapto-9-β-arabinofuranosylguanine compound to achieve the same level of yield as achieved with the Ni—Al alloy.

In the formulas of the present invention, R¹, R², R⁴, and R⁵ are each independently a hydrogen atom or a hydroxyl-protecting group. The hydroxyl-protecting group is not particularly limited and the substituents described in Protecting Groups in Organic Synthesis 3rd edition (Wiley-Interscience, Inc. 1999), which is incorporated herein by reference, can be used. Specifically, for example, acyl group, alkyl group, aralkyl group, silyl group, and the like can be mentioned. As the acyl group, for example, an acyl group having 1 to 7 carbon atoms, such as a formyl group, acetyl group, isobutyryl group, pivaloyl group, benzoyl group, and the like can be mentioned. As the alkyl group, for example, an alkyl group having 1 to 7 carbon atoms, such as a methyl group, ethyl group, n-prolyl group, isopropyl group, n-butyl group, tert-butyl group, and the like can be mentioned. As the aralkyl group, for example, an aralkyl group having 7 to 22 carbon atoms, such as a benzyl group, 4-monomethoxybenzyl group, trityl group, 4-monomethoxytrityl group, 4,4′-dimethoxytrityl group, and the like can be mentioned. As the silyl group, for example, a tri-substituted silyl group such as a trimethylsilyl group, triethylsilyl group, tert-butyldimethylsilyl group, and the like can be mentioned. In addition, a hydroxyl-protecting group such as a methoxymethyl group, methylthiomethyl group, benzyloxymethyl group, methoxyethoxymethyl group, tetrahydropyranyl group, methoxycarbonyl group, 9-fluorenylmethoxycarbonyl group, 2,2,2-trichloroethoxycarbonyl group, benzyloxycarbonyl group, tert-butoxycarbonyl group, and the like can be used. Since the reaction is carried out under basic conditions, a trityl group, 4-monomethoxytrityl group, 4,4′-dimethoxytrityl group, and a hydrogen atom are preferable, and a hydrogen atom is particularly preferable as R¹, R², R⁴, or R⁵.

In the formulas of the present invention, R³ and R⁶ are each a hydrogen atom or an amino-protecting group. The amino-protecting group is not particularly limited and the substituents described in Protecting Groups in Organic Synthesis 3rd edition (Wiley-Interscience, Inc. 1999), which is incorporated herein by reference, can be used. Specifically, alkyl group, aralkyl group, acyl group, alkoxycarbonyl group, alkylidene group and the like can be mentioned. As the alkyl group, for example, an alkyl group having 1 to 10 carbon atoms such as a methyl group, ethyl group, n-propyl group, isopropyl group, n-butyl group, tert-butyl group, and the like can be mentioned. As the aralkyl group, for example, an aralkyl group having 7 to 22 carbon atoms such as a benzyl group, 4-monomethoxybenzyl group, trityl group, 4-monomethoxytrityl group, 4,4′-dimethoxytrityl group, and the like can be mentioned. As the acyl group, for example, an acyl group having 1 to 10 carbon atoms such as a formyl group, acetyl group, isobutyryl group, pivaloyl group, benzoyl group, phenacyl group, and the like can be mentioned. As the alkoxycarbonyl group, a benzyloxycarbonyl group, tert-butoxycarbonyl group, methoxycarbonyl group, and the like can be mentioned. As the alkylidene group, a benzylidene group, diphenylmethylidene group, bis(methylthio)methylidene group, dimethylaminomethylidene group, and the like can be mentioned. Since reaction is carried out under basic conditions, a trityl group, 4-monomethoxytrityl group, 4,4′-dimethoxytrityl group, and a hydrogen atom are preferable, and a hydrogen atom is particularly preferable as R³ or R⁶.

While the 8,2′-anhydro-8-mercapto-9-β-arabinofuranosylguanine compound has enantiomers, a D-form, an L-form, or a mixture thereof may be used. An 8,2′-anhydro-8-mercapto-9-β-arabinofuranosylguanine compound can be produced according to the method described in Chem. Pharm. Bull., 1972, vol. 20(3), pp. 635-637, as shown in Scheme II, below. Specifically, guanosine (I) is first reacted with bromine to synthesize 8-bromoguanosine (II). Then, 8-bromoguanosine (II) is reacted with thiourea to synthesize 8-mercaptoguanosine (III), after which 8-mercaptoguanosine (III) is reacted with diphenyl carbonate to give 8,2′-anhydro-8-mercapto-9-β-arabinofuranosylguanine (IV). Protecting group(s) may be introduced into the hydroxyl group and amino group of 8,2′-anhydro-8-mercapto-9-β-arabinofuranosylguanine (IV) to give 8,2′-anhydro-8-mercapto-9-β-arabinofuranosylguanine compound (1). As the reagents for introducing protecting groups, any compounds can be used without any particular limitation as long as they are capable of affording protecting groups represented by R¹, R², and R³. For example, an acid anhydride such as acetic anhydride etc., an acid chloride such as acetyl chloride etc., and a silane compound such as t-butyldimethylsilyl chloride etc. can be mentioned. To prevent release of the protecting group, an acid anhydride is preferable and acetic anhydride is more preferable. 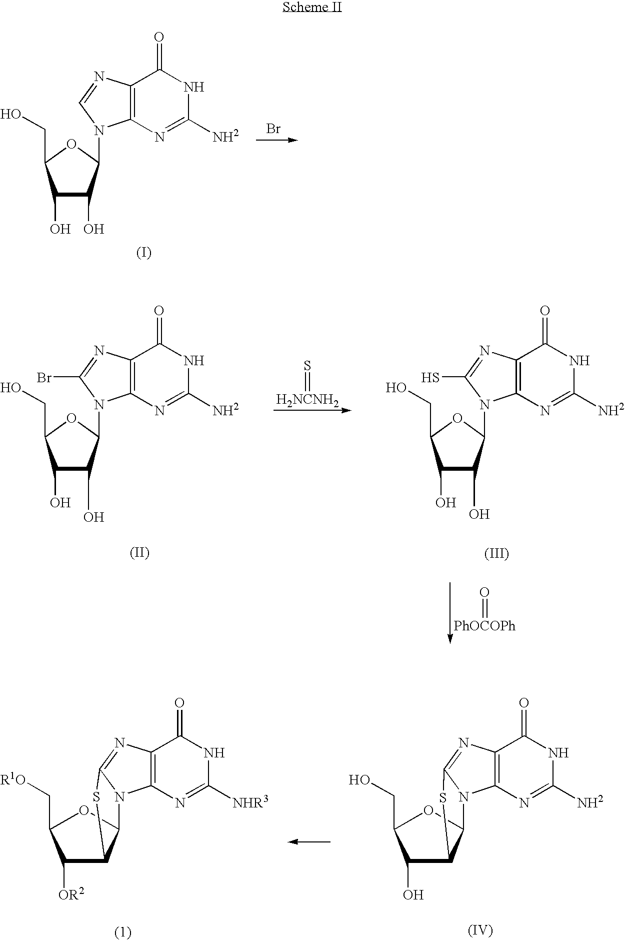
wherein R¹, R² and R³ are as defined above.

The 2′-deoxyguanosine compound represented by the formula (2) wherein one or more of R⁴, R⁵, and R⁶ of the compound are hydrogen atoms may be converted into a 2′-deoxyguanosine compound represented by the following formula (3) by replacing one or more of the hydrogen atoms with a protecting groups (protecting one or more of hydroxyl group and amino group with protecting groups).

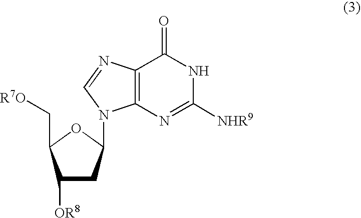

In the above-mentioned formula, R⁷ and R⁸ are each independently a hydrogen atom or a hydroxyl-protecting group and R⁹ is a hydrogen atom or an amino-protecting group, provided that R⁷, R⁸, and R⁹ are not simultaneously hydrogen atoms. As the hydroxyl-protecting group and amino-protecting group in this case, those similar to the ones mentioned above can be mentioned. Particularly, as the protecting groups for hydroxyl group and amino group, an acyl group is preferable and an isobutyryl group is more preferable.

As the nickel alloy, a Ni—Al alloy is preferable. As the Ni—Al alloy, one having a nickel and aluminum mass ratio of Ni:Al of 50 to 40:50 to 60 is preferable to allow easy development under basic conditions. The Ni—Al alloy may contain other metal elements (e.g., iron, molybdenum) as necessary. As the nickel, Raney-nickel obtained by treating a nickel alloy such as Ni—Al alloy etc. with sodium hydroxide and washing same with water is preferably mentioned.

In the present invention, the desulfurization reaction is carried out in the presence of a base. As the base, sodium hydroxide, potassium hydroxide, lithium hydroxide, sodium carbonate, potassium carbonate, lithium carbonate, ammonia, ammonium hydroxide, and the like can be mentioned, and sodium hydroxide, potassium hydroxide, and lithium hydroxide are particularly preferable.

The desulfurization reaction is carried out in a solvent in the present invention. As the solvent, water, methanol, ethanol, dimethyl sulfoxide, N,N-dimethylformamide, hexamethylphosphoric triamide, 2-methoxyethanol, ethylene glycol, or a mixed solvent thereof can be used. As preferable solvent, water, methanol, and ethanol can be mentioned, and water is particularly preferable. When the desulfurization reaction is carried out in an aqueous solvent, the pH of the aqueous solution before the desulfurization reaction is preferably set to not less than 11, more preferably not less than 12, to efficiently produce Raney-nickel from the Ni—Al alloy. It is desirable to prepare an aqueous solution to be used for the desulfurization reaction, for example, by adding a strong base such as sodium hydroxide, potassium hydroxide, lithium hydroxide etc. in water as a solvent to adjust the pH as mentioned above. In the production method of the present invention, it is preferable to dissolve an 8,2′-anhydro-8-mercapto-9-β-arabinofuranosylguanine compound in an aqueous solution and develop the Ni—Al alloy to Raney-nickel in the reaction system, under such conditions, thereby simultaneously carrying out the desulfurization reaction.

The desulfurization reaction can also be carried out under the ambient atmosphere. However, since nickel is easily oxidized by oxygen, the reaction is desirably carried out under an atmosphere of inert gas such as helium gas, argon gas, nitrogen gas, and the like. Preferable reaction conditions of the desulfurization reaction are as follows. The reaction temperature is preferably 40 to 100° C., more preferably 60 to 100° C. When it is less than 40° C., the reactivity and reaction rate tend to decrease. The reaction time is preferably 1 to 60 hours, more preferably 1 to 20 hours. When it is less than 1 hour, the desulfurization reaction tends to be insufficient.

When nickel such as Raney-nickel etc. is used as the nickel reagent, the amount thereof to be used is preferably 50 to 1500 mass %, more preferably 500 to 1500 mass %, relative to the total mass of the 8,2′-anhydro-8-mercapto-9-β-arabinofuranosylguanine compound. When it is less than 500 mass %, the reactivity and reaction rate tend to decrease, and even when it is used in excess of 1500 mass %, an effect comparable to the amount of addition may not be obtained. When Ni—Al alloy is used, the amount thereof to be used is preferably 50 to 500 mass %, more preferably 150 to 500 mass %, relative to the total mass of the 8,2′-anhydro-8-mercapto-9-β-arabinofuranosylguanine compound. When it is less than 150 mass %, the reactivity and reaction rate tend to decrease, and even when it is used in excess of 500 mass %, an effect comparable to the amount of addition may not be obtained. The “mass %” is expressed relative to the mass of the 8,2′-anhydro-8-mercapto-9-β-arabinofuranosylguanine compound as the standard (100 mass %). The mass of the Raney-nickel is the mass of Ni—Al alloy as a starting material, namely, the mass of Ni—Al alloy (starting material of Raney-nickel) before development to Raney-nickel.

The obtained crude 2′-deoxyguanosine compound can be purified by a known method such as crystal precipitation etc. As mentioned above, 2′-deoxyguanosine is subjected to tri-acylation or di-tritylation without separation and purification, and then analysis by high performance liquid chromatography (HPLC), whereby purity and yield of the 2′-deoxyguanosine compound can be confirmed.

analysis conditions of high performance liquid chromatography (HPLC):

column: Inertsil ODS-AQ (GL Science Inc.), 4.6×250 nm, room temp.

mobile phase: A) 0.02M AcOH—AcONH₄ (pH 3.5)
B) 0.02M AcOH—AcONH₄ (pH 3.5)/MeOH (60/40)
8% (0-5 min), 8-100% (5-47 min)

flow rate: 0.7 mL/min

detector: UV 260 nm

injection volume: 10 μL (sample/0.01M aqueous sodium hydroxide solution)

The retention time of the 2′-deoxyguanosine compound is 17.5 min.

In another embodiment, the present invention provides a method for producing an antisense drug by converting a 2′-deoxyguanosine compound represented by either formula (2) or (3) into an oligonucleotide, in which the 2′-deoxyguanosine compound represented by either formula (2) or (3) is prepared according to the present method. The conversion of the 2′-deoxyguanosine compound into an oligonucleotide may be carried out by any suitable method (see, e.g., Follmann H., Chem. Soc. Rev., 2004, vol. 33, pp. 225-233, which is incorporated herein by reference in its entirety).

Other features of the invention will become apparent in the course of the following descriptions of exemplary embodiments which are given for illustration of the invention and are not intended to be limiting thereof.

### EXAMPLES

1. Production of 2′-deoxyguanosine.

### Example 1

8,2′-Anhydro-8-mercapto-9-β-arabinofuranosylguanine (500 mg, 1.68 mmol), water (8.32 mL), 2M aqueous sodium hydroxide solution (1.68 mL), and Raney-nickel (7.5 g, Kawaken Fine Chemicals Co., Ltd., NDHT-M0) were added to a pear-shaped flask (50 mL), and the reaction mixture (pH 13.5) was stirred at 95° C. for 15 hours. The reaction mixture was filtered through a Kiriyama funnel under reduced pressure. The obtained solution was adjusted to pH 7 with 2M aqueous hydrochloric acid solution, and the neutralized solution was concentrated under reduced pressure. The crystals which precipitated during concentration were filtered through a Kiriyama funnel under reduced pressure, and the obtained crystals were vacuum dried for one day to give 2′-deoxyguanosine in a yield of 52% (0.87 mmol). The presence of 2′-deoxyguanosine (0.26 mmol) in the filtrate after filtration under reduced pressure was confirmed by high performance liquid chromatography (HPLC). The isolated 2′-deoxyguanosine and 2′-deoxyguanosine contained in the filtrate were obtained in a combined yield of 67%. ¹H-NMR (DMSO-d₆):δ 2.15-2.20 (m, 1H), 2.45-2.55 (m, 1H), 3.45-3.60 (m, 2H), 3.80 (br, 1H), 4.43 (br, 1H), 4.95 (br, 1H), 5.25 (d, 1H, J=3.8 Hz), 6.11 (m, 1H), 6.47 (br, 1H), 7.90 (s, 1H).

### Example 2

8,2′-Anhydro-8-mercapto-9-β-arabinofuranosylguanine (100 mg, 0.34 mmol), water (5.0 mL), 2M aqueous sodium hydroxide solution (0.34 mL), and Raney-nickel (500 mg, Kawaken Fine Chemicals Co., Ltd., NDHT-M0) were added to a pear-shaped flask (50 mL), and the reaction mixture (pH 13.1) was stirred at 95° C. for 15 hours. The reaction mixture was filtered through Kiriyama funnel under reduced pressure. The obtained solution was analyzed by HPLC to confirm that 2′-deoxyguanosine was obtained in a yield of 24% (0.08 mmol). It was also confirmed that 8,2′-anhydro-8-mercapto-9-β-arabinofuranosylguanine (0.15 mmol, 45%), a starting material, was recovered.

### Example 3

A Ni—Al alloy powder (10 g, Kawaken Fine Chemicals Co., Ltd., NDH) was added to a two-necked pear shaped flask (200 mL), and the inside of the flask was purged with argon gas. 8,2′-Anhydro-8-mercapto-9-β-arabinofuranosylguanine (5 g, 16.8 mmol) was dissolved in 2M aqueous sodium hydroxide solution (110 mL), and the solution (pH 14.3) was slowly added dropwise to the reaction system. The reaction mixture was stirred at 60° C. for 15 hours. The mixture was filtered through a Kiriyama funnel under reduced pressure and washed with water (35 mL). The obtained solution was analyzed by HPLC to confirm that 2′-deoxyguanosine was obtained in the yield of 53% (8.93 mmol).

### Example 4

A Ni—Al alloy powder (200 mg, Kawaken Fine Chemicals Co., Ltd., NDH) was added to a two-necked pear shaped flask (50 mL), and the inside of the flask was purged with argon gas. 8,2′-Anhydro-8-mercapto-9-β-arabinofuranosylguanine (100 mg, 0.34 mmol) was dissolved in 2M aqueous sodium hydroxide solution (2.2 mL), and the solution (pH 14.3) was slowly added dropwise to the reaction system. The reaction mixture was stirred at 60° C. for 4 hours. The mixture was filtered through a Kiriyama funnel under reduced pressure and washed with water (20 mL). The obtained solution was analyzed by HPLC to confirm that 2′-deoxyguanosine was obtained in the yield of 44% (0.15 mmol). It was also confirmed that 8,2′-anhydro-8-mercapto-9-β-arabinofuranosylguanine (0.02 mmol, 5%), a starting material, was recovered.

2. Conversion of 2′-deoxyguanosine to N²,3′,5′-triisobutyryl-2′-deoxyguanosine.

### Example 5

An aqueous sodium hydroxide solution containing 2′-deoxyguanosine (443.6 mg, 1.66 mmol) obtained in Example 1 was added to a wide-mouthed pear shaped flask (100 mL). The mixture was adjusted to pH 7 using 2M hydrochloric acid at 0° C. The obtained white suspension was concentrated under reduced pressure by a rotary evaporator, and azeotropic operation with toluene (5 mL) was performed twice. Furthermore, azeotropic operation with pyridine (5 mL) was performed twice. Pyridine (8 mL) and chloroform (12 mL) were added, and the mixture was cooled to 0° C. Isobutyryl chloride (1.8 mL, 16.8 mmol) was added, and the mixture was stirred at 0° C. for 30 minutes and at room temperature for 15 hours. Water was added to the reaction system to quench the reaction. The reaction mixture was extracted 3 times with chloroform (40 mL). The organic layer was washed with 5% aqueous sodium hydrogen carbonate solution and further washed with water. The organic layer was filtered through a Kiriyama funnel to remove a white solid. The obtained organic layer was dried over magnesium sulfate, and the magnesium sulfate was removed by filtration through a Kiriyama funnel. The organic layer was concentrated under reduced pressure, and the residue was purified by column chromatography. The fraction was concentrated under reduced pressure to give N²,3′,5′-triisobutyryl-2′-deoxyguanosine (517.6 mg, 1.08 mmol, 65% (2′-deoxyguanosine standard)).

### INDUSTRIAL APPLICABILITY

According to the present invention, a 2′-deoxyguanosine compound can be obtained in a high yield from an 8,2′-anhydro-8-mercapto-9-β-arabinofuranosylguanine compound. Moreover, the amount of nickel reagent can be markedly reduced by carrying out the reaction using a nickel-aluminum alloy, as compared to the use of nickel such as Raney-nickel etc. In addition, a 2′-deoxyguanosine compound can be produced safely, because preparation of highly active Raney-nickel prior to the desulfurization reaction becomes unnecessary.

Although only some exemplary embodiments of this invention have been described in detail above, those skilled in the art will readily appreciated that many modifications are possible in addition to the exemplary embodiments without materially departing from the novel teachings and advantages of this invention. Accordingly, all such modifications are intended to be included within the scope of this invention.

All patents, patent publications, and other publications identified or referenced herein are incorporated herein by this reference in full and in their entireties.

Obviously, numerous modifications and variations of the present invention are possible in light of the above teachings. It is therefore to be understood that, within the scope of the appended claims, the invention may be practiced otherwise than as specifically described herein.

1. A method of producing a 2′-deoxyguanosine compound represented by formula (2) 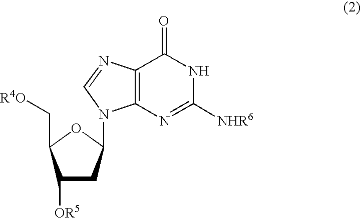
wherein:
R⁴ and R⁵ are each independently a hydrogen atom or a hydroxyl-protecting group, and
R⁶ is a hydrogen atom or an amino-protecting group,
which process comprises desulfurizing an 8,2′-anhydro-8-mercapto-9-β-arabinofuranosylguanine compound represented by formula (1) 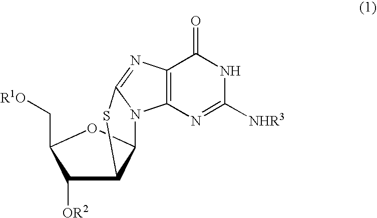
wherein:
R¹ and R² are each independently a hydrogen atom or a hydroxyl-protecting group, and
R³ is a hydrogen atom or an amino-protecting group,
wherein said desulfurizing is carried out in the presence of nickel or a nickel alloy, a solvent, and a base.

2. A method of producing a 2′-deoxyguanosine compound represented by formula (3) 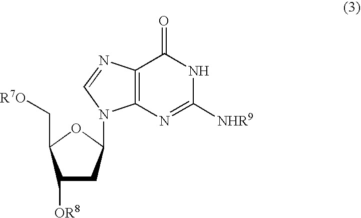
wherein:
R⁷ and R⁹ are each independently a hydrogen atom or a hydroxyl-protecting group, and
R⁹ is a hydrogen atom or an amino-protecting group,
provided that R⁷, R⁸ and R⁹ are not simultaneously hydrogen atoms,
which method comprises preparing a 2′-deoxyguanosine compound represented by the formula (2), in which one or more of R⁴, R⁵, and R⁶ of the compound are hydrogen atoms, according to the method of claim 1, and replacing one or more of said hydrogen atoms of R⁴, R⁵, and R⁶ with a protecting group.

3. The method of claim 1, wherein R¹, R², R³, R⁴, R⁵, and R⁶ are hydrogen atoms.

4. The method of claim 3, which further comprises a step of replacing each of said hydrogen atoms of R⁴, R⁵, and R⁶ with a protecting group.

5. The method of claim 1, wherein said solvent comprises water.

6. The method of claim 1, wherein said desulfurizing is carried out in a solution which comprises a base and a solvent and which has a pH of not less than 11.

7. The method of claim 1, wherein said nickel or nickel alloy is present in an amount of 50 to 500 mass % based on the total mass of the 8,2′-anhydro-8-mercapto-9-β-arabinofuranosylguanine compound at the start of said desulfurizing.

8. The method of claim 1, wherein said desulfurizing is carried out at a temperature of 40 to 100° C.

9. The method of claim 1, wherein said desulfurizing is carried out in the presence of a nickel-aluminum alloy.

10. The method of claim 1, wherein said desulfurizing is carried out in the presence of a base which is at least one member selected from the group consisting of sodium hydroxide, potassium hydroxide, lithium hydroxide, sodium carbonate, potassium carbonate, lithium carbonate, ammonia, ammonium hydroxide, and mixtures thereof.

11. The method of claim 1, wherein said desulfurizing is carried out in the presence of a base which is at least one member selected from the group consisting of sodium hydroxide, potassium hydroxide, lithium hydroxide, and mixtures thereof.

12. The method of claim 1, wherein said desulfurizing is carried out in the presence of a solvent which is at least one member selected from the group consisting of water, methanol, ethanol, dimethyl sulfoxide, N,N-dimethylformamide, hexamethylphosphoric triamide, 2-methoxyethanol, ethylene glycol, and mixtures thereof.

13. The method of claim 1, wherein said desulfurizing is carried out in the presence of a solvent which is at least one member selected from the group consisting of water, methanol, ethanol, and mixtures thereof.

14. The method of claim 1, wherein R¹, R², R⁴, and R⁵ are each independently a hydroxyl-protecting group and said hydroxyl-protecting group is selected from the group consisting of formyl group, acetyl group, isobutyryl group, pivaloyl group, benzoyl group, methyl group, ethyl group, n-propyl group, isopropyl group, n-butyl group, tert-butyl group, benzyl group, 4-monomethoxybenzyl group, trityl group, 4-monomethoxytrityl group, 4,4′-dimethoxytrityl group, trimethylsilyl group, triethylsilyl group, tert-butyldimethylsilyl group, methoxymethyl group, methylthiomethyl group, benzyloxymethyl group, methoxyethoxymethyl group, tetrahydropyranyl group, methoxycarbonyl group, 9-fluorenylmethoxycarbonyl group, 2,2,2-trichloroethoxycarbonyl group, benzyloxycarbonyl group, and tert-butoxycarbonyl group.

15. The method of claim 1, wherein R¹, R², R⁴, and R⁵ are each independently a hydroxyl-protecting group and said hydroxyl-protecting group is selected from the group consisting of trityl group, 4-monomethoxytrityl group, and 4,4′-dimethoxytrityl group.

16. The method of claim 1, wherein R³ and R⁶ are each an amino-protecting group and said amino-protecting group is selected from the group consisting of methyl group, ethyl group, n-propyl group, isopropyl group, n-butyl group, tert-butyl group, benzyl group, 4-monomethoxybenzyl group, trityl group, 4-monomethoxytrityl group, 4,4′-dimethoxytrityl group, formyl group, acetyl group, isobutyryl group, pivaloyl group, benzoyl group, phenacyl group, benzyloxycarbonyl group, tert-butoxycarbonyl group, methoxycarbonyl group, benzylidene group, diphenylmethylidene group, bis(methylthio)methylidene group, dimethylaminomethylidene group.

17. The method of claim 1, wherein R³ and R⁶ are each an amino-protecting group and said amino-protecting group is selected from the group consisting of trityl group, 4-monomethoxytrityl group, and 4,4′-dimethoxytrityl group.

18. In a method of preparing an oligonucleotide, said method comprising converting a 2′-deoxyguanosine compound into said oligonucleotide, wherein said 2′-deoxyguanosine compound has a structure according to formula (2) 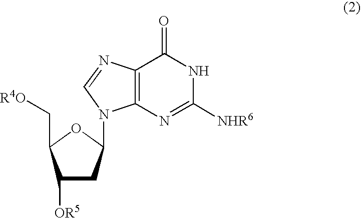
wherein:
R⁴ and R⁵ are each independently a hydrogen atom or a hydroxyl-protecting group, and
R⁶ is a hydrogen atom or an amino-protecting group,
the improvement being said 2′-deoxyguanosine compound having a structure according to formula (2) is prepared by the method of claim 1.

19. In a method of preparing an oligonucleotide, said method comprising converting a 2′-deoxyguanosine compound into said oligonucleotide, wherein said 2′-deoxyguanosine compound has a structure according to formula (3) 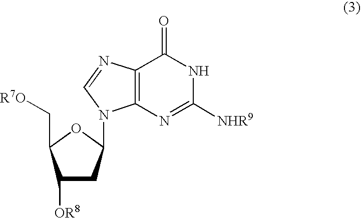
wherein:
R⁷ and R⁸ are each independently a hydrogen atom or a hydroxyl-protecting group, and
R⁹ is a hydrogen atom or an amino-protecting group,
provided that R⁷, R⁸ and R⁹ are not simultaneously hydrogen atoms,
the improvement being said 2′-deoxyguanosine compound having a structure according to formula (3) is prepared by the method of claim 2.
